(19) United States
(12) Patent Application Publication
(10) Pub. No.: US 2004/0049049 A1
(43) Pub. Date: Mar. 11, 2004

(54) Stereoselective synthesis of 1,2-disubstituted cycloalkyls

(75) Inventors: Jurjus F. Jurayj, Acton, MA (US); Emile Farhan, Dedham, MA (US); Pradeep K. Sharma, Westford, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Johnson Matthey Pharmaceutical Materials, Inc., Devens, MA

(21) Appl. No.: 10/458,799

(22) Filed: Jun. 10, 2003

Related U.S. Application Data
(60) Provisional application No. 60/389,418, filed on Jun. 14, 2002.

(51) Int. Cl. C07D 213/36; C07D 207/30

(52) U.S. Cl. 546/329; 548/561; 562/402

(57) ABSTRACT

A stereoselectively method of preparing a 1,2-disubstituted cycloalkyl, such as aminocycloalkyl ether compounds, from a trans-1,2-disubstituted cycloalkyl or a cis-2-substituted cycloalkanol. For example, a stereoselective method of preparing 1R-(3R-hydroxypyrrolidin-1-yl)-2R-(2-phenylethoxy)-cyclohexane from 1R,2R-cyclohexanediol or from meso-cis-1,2-cyclohexanediol is described. Aminocycloalkyl ethers, such as 1R-(3R-hydroxypyrrolidin-1-yl)-2R-(2-phenylethoxy)-cyclohexane, can be used to treat cardiac disease.

### RELATED APPLICATION

This application claims the benefit of U.S. Provisional No. 60/389,418, filed Jun. 14, 2002. The entire teaching of the above application is incorporated herein by reference.

### BACKGROUND OF THE INVENTION

Conservative estimates indicate that, in the U.S. alone, approximately 300,000 individuals per year suffer heart attacks. Approximately half of these die from sudden cardiac death, the major cause of which is ventricular fibrillation, a type of cardiac arrhythmia.

1,2-Disubstituted cycloalkyls, such as the aminocycloalkyl ether compounds disclosed in WO 99/50225 and WO 00/47547, have been shown to be effective in treating cardiac disease, such as cardiac arrhythmias. However, the methods of synthesizing aminocycloalkyl ethers provided in WO 99/50225 and WO 00/47547 lead to a mixture of stereoisomers. It is often desirable to obtain a stereochemically pure form of a pharmaceutically active compound because pharmaceuticals which interact with a specific target are often more potent and/or have less deleterious side effects when they are administered in their stereochemically pure form. Separation of stereoisomers after synthesis is often difficult, if not impossible. In addition, separation of isomers leads to waste since a portion of the product has the wrong stereochemistry and must be discarded. Therefore, a need exists for a method to form stereochemically pure 1,2-disubstituted cycloalkyls that overcome or minimize the problems discussed above.

### SUMMARY OF THE INVENTION

The invention is a method of stereoselectively preparing a 1,2-disubstituted cycloalkane represented by Structural Formula I:

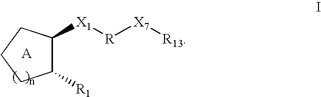

In Structural Formula I, ring A is substituted or unsubstituted; n is 1, 2, or 3; X, is —O—, —S—, or —NR₂—; X₇ is a bond, —O—, —S—, or —CR₂₀═CR₂₁—; R is an alkylene group; R₁ is —OR₃, —SR₃ or —NR₄R₅; R₁₃ is an aliphatic group, an aryl group or a heteroaryl group; R₂ and R₃ are each, independently, —H, an aliphatic group, an aliphatic carbonyl, an aralkyl, an aryl, a heteroaryl, a heteroaralkyl, a heterocycloalkyl, or a group represented by the formula —Y—R₆, wherein Y is an alkylene group and R₆ is a heterocycloalkyl group; and R₄ and R₅ are each, independently, —H, an aliphatic group, an aliphatic carbonyl, an aralkyl, an aryl, a heteroaryl, a heteroaralkyl, a heterocycloalkyl, or a group represented by the formula —Y—R₆; or R₄ and R₅ together with the nitrogen to which they are attached are a heteroaryl or a heterocycloalkyl; R₂₀ and R₂₁ are each, independently, —H, an aliphatic group, an aryl group, or an aralkyl. The method involves reacting a trans-1R,2R-disubstituted cycloalkane represented by Structural Formula II:

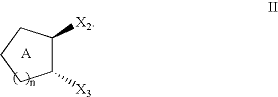

with a compound having a leaving group represented by Structural Formula III:

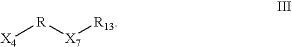

to form a compound represented by Structural Formula W:

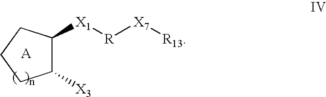

In Structural Formula II, X₂ is —OH, —SH, or —NHR₂; X₃ is —OH, a protected alcohol, or a halo; and n is defined as in Structural Formula I. In Structural Formula III, X₇, R, and R₁₃ are defined as in Structural Formula I, and X₄ is a leaving group. Leaving groups that can be used in the above reaction include halogens, —OSO₂-aryl, —OSO₂— (aliphatic group), and 2,2,2-trihaloacetimidate. In Structural Formula IV, X₁, X₇, R, R₁₃ and n are defined as in Structural Formula I, and X₃ is defined as in Structural Formula II.

A compound represented by Structural Formula IV is reacted with a carboxylic acid in the presence of triphenyl phosphine and dialkyl azodicarboxylate or a halogen source to form a compound represented by Structural Formula V. When the compound represented by Structural Formula IV is reacted with a carboxylic acid, the ester formed is hydrolyzed with, for example NaOH, to form a hydroxy group and the hydroxy group is reacted with a compound selected from the group consisting of X—SO₂-aryl, X—SO₂— (aliphatic group), and 2,2,2-trihaloacetonitrile. The compound formed by reaction with the halogen source or the carboxylic acid is represented by Structural Formula V:

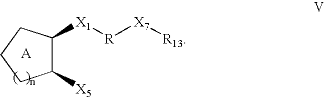

In Structural Formula V, X₅ is a halo, —OSO₂-aryl, —OSO₂— (aliphatic group), or 2,2,2-trihaloacetimidate; and X₁, X₇, R, R₁₃ and n are defined as in Structural Formula I. In general, the reaction is done under conditions that promote S_{N}2 substitution while minimizing S_{N}1 substitution.

A compound represented by Structural Formula V, is reacted with a nucleophile selected from the group consisting of HR₁ or M⁺⁻R₁, wherein M⁺ is a metal cation, such as Na⁺, Li⁺ or K⁺, to form a compound represented by Structural Formula I. In general, this reaction is also carried out under conditions that promote S_{N}2 substitution. For example, the nucleophile should be a strong base or deprotonated by a strong base. When the nucleophile is HR₁, a strong base, such as NaH, a trialkyl amine, 1,8-diazabicyclo[5,4.0]undec-7-ene (DBU) and the like, can be used to deprotonate HR₁ and thus facilitate S_{N}2 reaction. Non-polar solvents may also be used to minimize S_{N}1 reaction.

In an alternative embodiment, compounds represented by Structural Formula I are stereoselectively prepared by reacting a cis-2-substituted cycloalkanol represented by Structural Formula VI:

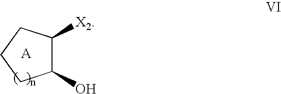

with a galactose derivative represented by Structural Formula VII:

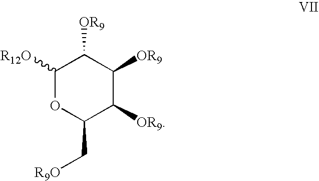

to form a galactose-substituted cycloalkanol represented by Structural Formula VIII:

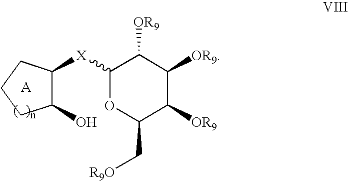

In Structural Formula VI, X₂ is defined as in Structural Formula II. In Structural Formulas VI and VIII, n is defined as in Structural Formula I. In Structural Formula VIII, X₁ is defined as in Structural Formula I. In Structural Formulas VII and VIII, R₉ for each occurrence is, independently, —H or an alcohol protecting group. Alternatively, two adjacent —OR groups together with the carbon atoms to which they are attached form a [1,3]dioxolane. In a preferred embodiment, each R₉ is —H in Structural formula VII. R₁₂ is an aryl, a cycloalkyl, or a heterocycloalkyl. The reaction is carried out in the presence of an enzyme that stereoselectively catalyzes addition of galactose to the hydroxy, thio, or amine group at the carbon having an R-configuration in Structural Formula VI. A preferred enzyme is β-galactosidase. In general, the solvent for the reaction is water, a water miscible solvent (e.g., tetrahydrofuran, dioxane, an alcohol, dimethyl formamide, dimethyl sulfoxide, and the like), or a mixture of water and a water miscible solvent. After galactose-substituted cycloalkanol represented by Structural Formula VIII has been formed, two hydroxy groups on adjacent carbon atoms (1,2-diol) and/or two hydroxy groups on carbon atoms that are separated from each other by one carbon atom (1,3-diol) of the galactose substituent are protected with a cyclic acetal or a cyclic ketal. Methods for protecting 1,2-diols and 1,3-diols as cyclic acetals and cyclic ketals can be found in Greene, et al., Protective Groups in Organic Synthesis, (1991), John Wiley & Sons, Inc., pages 118-142, the teachings of which are incorporated herein by reference in their entirety. A preferred ketal for protecting 1,2-diols or 1,3-diols is isopropylidene ketal. Id., pages 123-127.

The compound represented by Structural Formula VIII is reacted with an alcohol protecting group to form a compound represented by Structural Formula IX:

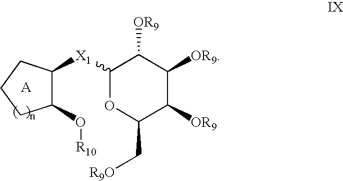

In Structural Formula IX, X₁ and n are defined as in Structural Formula I, R₉ is defined as in Structural Formula VII, and R₁₀ is an alcohol protecting group, such as a substituted or unsubstituted benzyl group. When a substituted or unsubstituted benzyl protecting group is used, a compound represented by Structural Formula VIII is reacted with, for example, a substituted or unsubstituted benzyl halide, such as benzyl chloride or benzyl bromide, in the presence of a base, such as potassium hydroxide.

The compound represented by Structural Formula IX is treated with an acid to form a protected cycloalkanol represented by Structural Formula X:

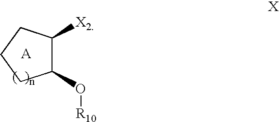

In Structural Formula X, n is defined as in Structural Formula I, X₂ is defined as in Structural Formula II, and R₁₀ is defined as in Structural Formula IX.

The compound represented by Structural Formula X is reacted with a compound represented by Structural Formula m to form a compound represented by Structural Formula XI:

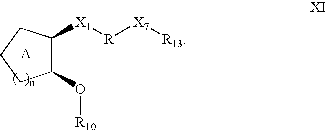

In Structural Formula XI, X₁, X₇, R, R₁₃, and n are defined as in Structural Formula I, and R₁₀ is defined as in Structural Formula IX.

The alcohol protecting group represented by R₁₀ in Structural Formula XI is removed to form a compound represented by Structural Formula XII:

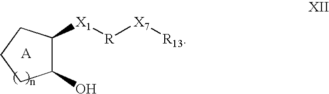

In Structural Formula XII, X₁, X₇, R, R₁₃, and n are defined as in Structural Formula I. When R₁₀ is a benzyl group, the benzyl group can be removed by treating a compound represented by Structural Formula XI with a catalytic amount of palladium in the presence of hydrogen gas. Typically, the reaction is carried out in a protic solvent, such as an alcohol, in a hydrogen atmosphere. When R₁ of Structural Formula I is —SR₃, it is desirable to avoid using a palladium catalyst to remove the benzyl protecting group because the palladium will poison the sulfide nucleophile used to displace the activated alcohol in the next reaction step. In this case, the benzyl protecting group can be removed by an alternative method, such as treatment with iodotrimethylsilane in acetonitrile or Ph₃C⁺BF₄⁻ in CH₂Cl₂. For other methods of cleaving benzyl protecting groups see Greene, Protective Groups in Organic Synthesis, (1999), John Wiley & Sons, Inc., pages 86-112. Alternatively, an acid stable alcohol protecting group other than a benzyl group can be used.

A compound represented by Structural Formula XII is reacted with an alcohol activating agent and a nucleophile selected from the group consisting of HR₁ or M⁺⁻R₁, wherein M⁺ is a metal cation, to form a compound represented by Structural Formula I. Typical, alcohol activating agents that can be used in this reaction include X—SO₂-aryl, for example tosyl chloride or tosyl bromide, X—SO₂— (aliphatic group), for example mesyl chloride, mesyl bromide or trifluromethanesulfonyl chloride, and 2,2,2-trihaloacetonitrile, for example trifluoroacetimidoyl chloride, wherein X is a halo, to form an activated alcohol. In general, the alcohol group is reacted with the alcohol activating agent in the presence of an aprotic base such as a trialkyl amine, pyridine or DBU. Then the activated alcohol is displaced by HR₁ or M⁺⁻R₁ under conditions that promote S_{N}2 substitution while minimizing S_{N}1 substitution.

In another alternative embodiment, compounds represented by Structural Formula I can be stereoselectively prepared by reacting a galactose substituted cycloalkanol represented by Structural Formula VIII with an alcohol activating agent and a nucleophile selected from the group consisting of HR₁ or M⁺⁻R₁, wherein M⁺ is a metal cation, to form a compound represented by Structural Formula XIII:

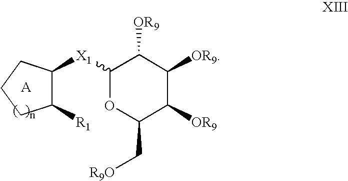

In Structural Formula XIII, X₁, R₁, and n are defined as in Structural Formula I, and R₉ is defined as in Structural Formula VII. In a preferred embodiment, two hydroxy groups on adjacent carbon atoms (1,2-diols) and/or two hydroxy groups on carbon atoms that are separated from each other by one carbon atom (1,3-diols) of the galactose substituted cycloalkanol represented by Structural Formula VIII are protected with a cyclic acetal or a cyclic ketal prior to reacting the galactose substituted cycloalkanol with the alcohol activating agent and the nucleophile.

A compound represented by Structural Formula XIII is treated with an acid to form a compound represented by Structural Formula XIV:

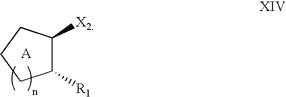

In Structural Formula XIV, R₁ and n are defined as in Structural Formula I, and X₂ is defined as in Structural Formula II.

The compound represented by Structural Formula XIV is reacted with a compound represented by Structural Formula III to form a compound represented by Structural Formula I.

In another alternative embodiment, compounds represented by Structural Formula I can be stereoselectively prepared by reacting a cis-1R-substituted-2S-halo-cycloalkyl represented by Structural Formula XV:

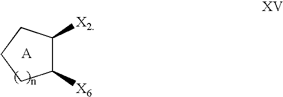

with a compound represented by Structural Formula III to form a compound represented by Structural Formula XVI:

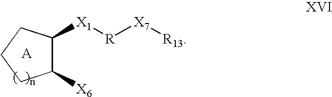

In Structural Formula XV, X₂ is defined as in Structural Formula II, n is defined as in Structural Formula I, and X₆ is a halo. In Structural Formula XVI, X₁, X₇, R, R₁₃, and n are defined as in Structural Formula I, and X₆ is defined as in Structural Formula XV.

A compound represented by Structural Formula XVI is reacted with HR₁ or M⁺⁻R₁, wherein M⁺ is a metal cation, to form a compound represented by Structural Formula I. In general, this reaction is also carried out under conditions that promote S_{N}2 substitution while minimizing S_{N}1 substitution.

The method of the invention provides a stereoselective route to 1,2-disubstituted cycloalkyl compounds, such as aminocycloalkyl ether compounds. Thus, stereochemically pure stereoisomers of 1,2-disubstituted cycloalkyl compounds can be obtained by the method of the invention while avoiding, or reducing the difficulty of separating stereoisomers.

### DETAILED DESCRIPTION OF THE INVENTION

The term “aliphatic” as used herein refers to optionally substituted straight-chain, branched or cyclic C₁-C₁₂ hydrocarbons which are completely saturated or which contain one or more units of unsaturation but which are not aromatic. For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl. The terms “alkyl,” “alkoxy,” and “alkylthio,” used alone or as part of a larger moiety includes both straight and branched chains containing one to twelve carbon atoms. The terms “alkenyl” and “alkynyl” used alone or as part of a larger moiety includes both straight and branched chains containing two to twelve carbon atoms. The term “cycloalkyl” used alone or as part of a larger moiety includes cyclic C₃-C₁₂ hydrocarbons which are completely saturated or which contain one or more units of unsaturation, but which are not aromatic.

The term “alkylene,” as used herein, refers to optionally substituted divalent aliphatic group that has two points of attachments. Preferably, alkylene groups are divalent alkyl group having two points of attachments, such as —CH₂—, —CH₂CH₂—, —CH(CH₃)CH₂—, and the like.

As used herein, aryl groups are optionally substituted carbocyclic aromatic ring systems (e.g. phenyl), optionally substituted fused polycyclic aromatic ring systems (e.g. naphthyl and anthracenyl) and optionally substituted aromatic ring systems fused to optionally substituted carbocyclic non-aromatic ring systems (e.g., 1,2,3,4-tetrahydro-naphthyl and indanyl) having six to about fourteen carbon atoms. The term “aryl” used alone or as part of a larger moiety as in “aralkyl,” “aralkoxy,” or “aryloxyalkyl,” refers to aromatic ring groups having six to fourteen members, such as phenyl, benzyl, phenethyl, 1-napthyl, 2-naphthyl, 1-anthracyl and 2-anthracyl. The term “aryl” may be used interchangeably with the term “aryl ring.”

The terms “haloalkyl,” “haloalkenyl,” and “haloalkoxy” means alkyl, alkenyl or alkoxy, as the case may be, substituted with one or more halogen atoms.

The term “halo” or “halogen” means F, Cl, Br or I.

The term “heteroatom” means nitrogen, oxygen, or sulfur and includes any oxidized form of nitrogen and sulfur, and the quaternized form of any basic nitrogen. When a nitrogen atom is part of a heterocycloalkyl or heteroaryl ring, it can be substituted or unsubstituted. For example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR₁, (as in N-substituted pyrrolidinyl). R₁₁ is a substituent. Examples of substituents encompassed by R₁₁ are described below.

The term “heterocycloalkyl,” as used herein refers to optionally substituted non-aromatic ring systems having three to fourteen members, preferably five to ten, in which one or more ring carbons, preferably one to four, are each replaced by a heteroatom such as N, O, or S. Examples of heterocycloalkyl rings include 3-1H-benzimidazol-2-one, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocycloalkyl,” as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycloalkyl,” whether saturated or partially unsaturated, also refers to rings that are optionally substituted.

The term “heteroaryl,” used alone or as part of a larger moiety as in “heteroaralkyl,” refers to optionally substituted heteroaromatic ring groups having five to fourteen members, wherein from one to about six members are heteroatoms. Examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 2-triazolyl, 5-triazolyl, 2-thienyl, 3-thienyl, thianaphthenyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, or benzoisazolyl. Also included within the scope of the term “heteroaryl,” as it is used herein, is a group in which an optionally substituted heteroatomic ring is fused to one or more optionally substituted aromatic or optionally substituted nonaromatic rings where the radical or point of attachment is on the heteroaromatic ring. Examples include tetrahydroquinolinyl, tetrahydroisoquinolinyl, and pyrido [3,4-d]pyrimidinyl.

An aralkyl group, as used herein, is an aryl substituent that is linked to a compound by an alkylene group having from one to twelve carbon atoms.

An heteroaralkyl group, as used herein, is a heteroaryl substituent that is linked to a compound by an alkylene group having from one to twelve carbon atoms.

An alkoxy group, as used herein, is a C₁-C₁₂ alkyl group that is connected to a compound via an oxygen atom. Examples of alkoxy groups include, but are not limited to, methoxy, ethoxy, propoxy, isopropoxy, and t-butoxy.

A aliphatic carbonyl group, as used herein, is an aliphatic group that is connected to a compound via a carbonyl group. A preferred aliphatic carbonyl is acetyl.

Alcohol protecting groups are known to those skilled in the art. For examples of alcohol protecting groups see Greene, et al., Protective Groups in Organic Synthesis, (1999), John Wiley & Sons, Inc., pages 17-245, the teachings of which are incorporated herein by reference in their entirety. A preferred alcohol protecting group is a benzyl group.

A “leaving group” is defined herein as a group that can be displaced by a nucleophile (e.g., —OH, —SH, or —NR₅R₆) to form a weak base. Examples of leaving groups include halo, —OSO₂— (substituted or unsubstituted aryl), —OSO₂— (substituted or unsubstituted alkyl), and 2,2,2-trihaloacetimidate.

Suitable halogen sources are a compound that provides halogen ions. Examples of suitable halogen sources include SOCl₂, PBr₃, POBr₃, PCl₃, POCl₃ and halide salts such as LiBr or LiCl.

An aryl group (e.g., ring B of Structural Formula I) or a heteroaryl group may contain one or more substituents. Examples of suitable substituents include aliphatic groups (including haloalkyl, such as trifluoromethyl and trichloromethyl), aryl groups, alkoxy groups, heteroaryl groups, heteroaralkyl groups, aralkyl groups, halo, hydroxy, —OR₁₄, —COR₁₄, —COOR₁₄, —NHCOR₁₄, —OCOR₁₄, benzyl, halo, cyano, nitro, —SO³⁻, —SH, —SR₁₄, —NH₂, —NHR₁₄, —NR₁₄R₁₅, —NR₁₄CO₂R₁₅, —NR₁₄R₁₅C(O)N(R₁₆)₂, —C(O)CH₂C(O)R₁₄, —CO₂R₁₄, —C(O)R₁₄, —C(O)N(R₁₄)₂, —OC(O)N(R₁₄)₂, —S(O)₂R₁₄, —SO₂N(R₁₄)₂, —S(O)R₁₄, —C(═S)N(R₁₄)₂, —C(═NH)—N(R₁₄)₂, and COOH, wherein R₁₄, R₁₅ and R₁₆ are each, independently, an aliphatic group, an aryl group, or an aralky group.

A heterocycloalkyl, aliphatic group (e.g., ring A of Structural Formula I), or an alkylene group may contain one or more substituents. Examples of suitable substituents on a saturated or unsaturated carbon of a heterocycloalkyl, aliphatic group or alkylene group include those listed above for an aryl and heteroaryl groups. In addition, the following groups can be a substituent on a saturated carbon of a heteroaryl, aliphatic group or alkylene group: ═O, ═S, ═NNHR₁₇, ═NN(R₁₇)₂, ═NNHC(O)R₁₇, ═NNHCO₂(alkyl), ═NNHSO₂(alkyl), or ═NR₁₇, where each R₁₇ is independently selected from hydrogen, an aliphatic group.

Suitable substitutents on the nitrogen of a non-aromatic heterocycloalkyl or on an unsaturated nitrogen of a heteroaryl include —R₁₈, —N(R₁₈)₂, —C(O)R₁₈, —CO₂R₁₈, —C(O)CH₂C(O)R₁₈, —C(═S)N(R₁₈)₂, and —C(═NH)N(R₁₈)₂; wherein R₁₈ is hydrogen, an aliphatic group, phenyl, substituted phenyl, benzyl, or a heteroaryl or heterocycloalky. Examples of substituents on R₁₈ when R₁₈ is an aliphatic group or a phenyl include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

The invention is a method of stereoselectively preparing a 1,2-disubstituted cycloalkyl represented by Structural Formula I. In one embodiment, trans-1R,2R-disubstituted cycloalkyl represented by Structural Formula II is reacted with a compound represented by Structural Formula III via a nucleophilic displacement reaction (e.g., S_{N}1 or S_{N}2 reaction) to form a compound represented by Structural Formula IV. In a preferred embodiment, R₁₃ of Structural Formula III is a phenyl, naphthyl, indolyl, fluorenyl, or acenaphthyl. Typically, the leaving group represented by X₄ is a halo, —OSO₂-aryl, such as tosyl, —OSO₂— (aliphatic group), such as mesyl or triflate, and 2,2,2-trihaloacetimidate, such as 2,2,2-trichloroacetimidate or 2,2,2-trifluoroacetimidate.

The compound represented by Structural Formula IV is reacted with a halogen source to form a cis-1,2-disubstituted cycloalkyl represented by Structural Formula V in which X₅ is a halo. Alternatively, the compound represented by Structural Formula IV is reacted with a carboxylic acid to form an ester. The ester is then hydrolyzed to form an alcohol which can be reacted with an alcohol activating agent, such as X—SO₂-aryl, X—SO₂— (aliphatic group), or 2,2,2-trihaloacetonitrile (see, for example, Scheme I) to form a compound represented by Structural Formula V in which X₅ is —OSO₂-aryl, —OSO₂— (aliphatic group) or 2,2,2-trihaloacetimidate.

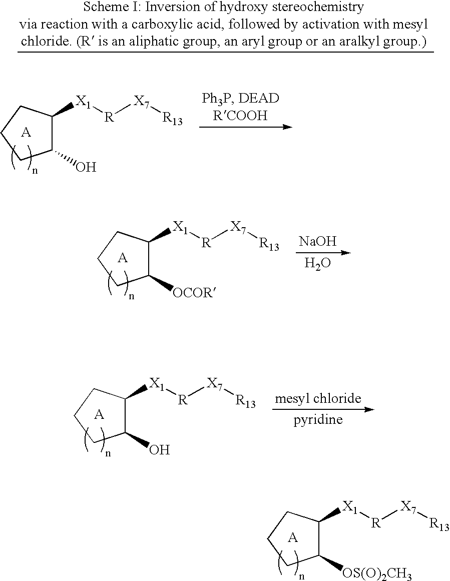

When X₃ of the compound represented by Structural Formula IV is —OH, the halogen source is typically SOCl₂ in pyridine or a diarylchlorophosphite followed by HBr. When X₃ is a protected alcohol, the alcohol protecting group is removed first, and the free alcohol group is reacted with SOCl₂ in pyridine or a diarylchlorophosphite followed by HBr.

In an alternative embodiment, when X₃ is —OH, the reaction with the halogen source involves two steps. In the first step, the compound represented by Structural Formula V is reacted with a compound selected from the group consisting of X—SO₂-aryl, X—SO₂— (aliphatic group), and 2,2,2-trihaloacetonitrile, wherein X is a halo, to form an activated alcohol. This reaction is typically carried out in the presence of an aprotic base such as pyridine, trialkyl amine, or DBU. In the second step, the activated alcohol is reacted with a halide salt, such as LiCl or LiBr. When X₃ is a protected alcohol, the alcohol protecting group is removed first, and the free alcohol group is reacted with X—SO₂-aryl, X—SO₂— (aliphatic group), and 2,2,2-trihaloacetonitrile.

The compound represented by Structural Formula V is then reacted with a nucleophile selected from the group consisting of HR, or M⁺⁻R₁. In a preferred embodiment, the nucleophile is HNR₄R₅. In a more preferred embodiment, the nucleophile is represented by Structural Formula XVII:

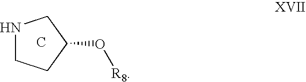

In Structural Formula XVII, R₈ is —H or an alcohol protecting group; and ring C is substituted or unsubstituted.

In one embodiment, R₈ is —H.

In another embodiment, the compound having leaving group X₄ is a compound represented by Structural Formula XVIII:

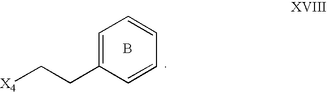

In Structural Formula XVIII, X₄ is defined as in Structural Formula III.

In another embodiment, R₈ is —H, n is 2, X₁ is —O—, X₂ is —OH and the 1,2-disubstituted cycloalkyl formed is the compound prepared is represented by Structural Formula XVII:

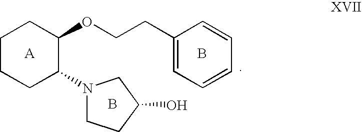

Preferably, the compound represented by Structural Formula (XVII) is 1R-(3R-hydroxypyrrolidin-1-yl)-2R-(2-phenylethoxy)-cyclohexane.

In an alternative embodiment, a 1,2-disubstituted cycloalkyl represented by Structural Formula I is stereoselectively prepared from a cis-2-substituted-cycloalkanol represented by Structural Formula VI. The compound represented by Structural Formula VI is reacted with a galactose derivative represented by Structural Formula VII in the presence of β-galactosidase. β-galactosidase preferentially catalyzes the reaction of X₂ of the compound represented by Structural Formula VI (i.e., the nucleophilic substituent on the carbon having an R-configuration) with the galactose derivative to form a galactose substituted cycloalkanol represented by Structural Formula VIII. The reaction is typically carried out in water, a water miscible solvent, or a mixture of water and a water miscible solvent.

Reaction of the 2R-substituent with the galactose derivative allows the alcohol group having the S-configuration to be selectively protected with an alcohol protecting group to form a compound represented by Structural Formula IX. In one embodiment, the alcohol is protected with a benzyl group.

The galactose substituent is then removed by treating the compound represented by Structural Formula IX with an acid to form a compound represented by Structural Formula X. Typically, the galactose group is removed by treating the compound represented by Structural Formula IX with HCl in an alcohol.

The compound represented by Structural Formula X is reacted with a compound represented by Structural Formula III via a nucleophilic displacement reaction (e.g., S_{N}1 or S_{N}2 reaction) to form a compound represented by Structural Formula XI.

R₁₀ is removed to form a compound represented by Structural Formula XII. When R₁₀ is a benzyl group, it is typically removed by reacting the compound represented by Structural Formula XI with hydrogen gas in the presence of palladium on carbon and a protic solvent.

The compound represented by Structural Formula XII is then reacted with an alcohol activating agent and a nucleophile selected from the group consisting of HR₁ or M⁺⁻R₁. In one embodiment, the reaction is a Mitsunobu reaction (Hugh, Org. Prep. Proced. Int., (1996), 28:127-164, the entire teachings of which are incorporated herein by reference). In a preferred embodiment, the nucleophile is HNR₄R₅ and the activating agent includes a dialkyl azodicarboxylate and triphenyl phosphine. More preferably, the nucleophile is a compound represented by Structural Formula XVII. Alternatively, the alcohol activating agent is selected from the group consisting of X—SO₂-aryl, X—SO₂— (aliphatic group), and 2,2,2-trihaloacetonitrile, wherein X is a halo, and the nucleophile is HNR₄R₅. More preferably, the nucleophile is a compound represented by Structural Formula XVII.

In another alternative embodiment, a 1,2-disubstituted cycloalkyl represented by Structural Formula I is stereoselectively prepared from a galactose substituted cycloalkanol represented by Structural Formula VIII. The galactose substituted cycloalkanol is reacted with an alcohol activating agent and a nucleophile selected from HR₁ and M⁺⁻R, to form a compound represented by Structural Formula XIII. In a preferred embodiment, the nucleophile is HNR₄R₅. More preferably, the nucleophile is a compound represented by Structural Formula XVII. When it is desirable to prevent wasting the nucleophile and/or the alcohol activating agent, the 1,2-diol and 1,3-diol groups of the galactose substituent of Structural Formula VIII are selectively protected as a cyclic acetal or a cyclic ketal before reaction with the alcohol activating agent and the nucleophile.

The galactose substituent is then removed by treating the compound represented by Structural Formula XIII with an acid to form a compound represented by Structural Formula XIV. Typically, the galactose group is removed by treating the compound represented by Structural Formula XIII with HCl in an alcohol.

The compound represented by Structural Formula XIV is reacted with a compound represented by Structural Formula III via a nucleophilic displacement reaction (e.g., S_{N}1 or S_{N}2 reaction) to form a compound represented by Structural Formula I.

In another alternative embodiment, a 1,2-disubstituted cycloalkyl represented by Structural Formula I is stereoselectively prepared from a 1R-substituted-2S-halo-cycloalkyl represented by Structural Formula XV. When X₂ of Structural Formula XV is —NHR₂, R₂ is preferably a group that decreases the nucleophilicity of the nitrogen, such as an aliphatic carbonyl group. The compound represented by Structural Formula XV is reacted with a compound represented by Structural Formula III via a nucleophilic displacement reaction (e.g., S_{N}1 or S_{N}2 reaction) to form a compound represented by Structural Formula XVI.

The compound represented by Structural Formula XVI is then reacted with a nucleophile selected from the group consisting of HR₁ or M⁺⁻R₁. In a preferred embodiment, the nucleophile is HNR₄R₅. More preferably, the nucleophile is a compound represented by Structural Formula XVII.

The following are examples of specific embodiments of the invention and are not intended to be limiting in any way.

### EXAMPLES

I. Method I for Preparing 1R-(3R-hydroxypyrrolidin-1-yl)-2R-(2-phenylethoxy)-cyclohexane.

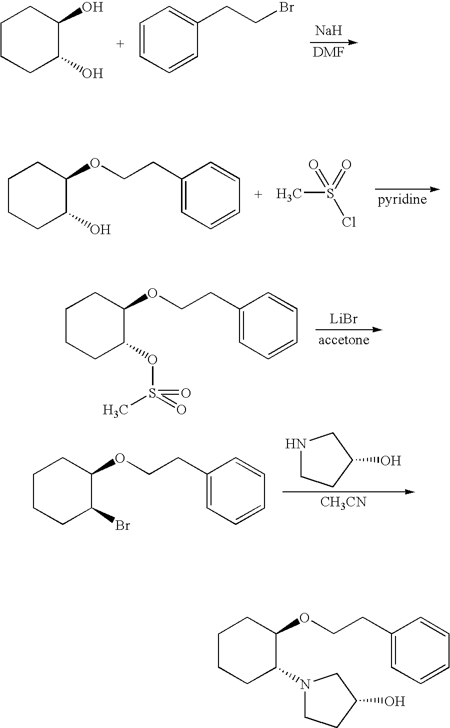

Scheme II: Method I for Preparing 1R-(3R-hydroxypyrrolidin-1-yl)-2R-(2-phenylethoxy)-cyclohexane.

The starting material, 1R,2R-cyclohexanediol, is commercially available or can be prepared by enzymatic hydrolysis of the racemic diacetate of cyclohexandiol (Faber, K., “Biotransformation in Organic Chemistry: a Textbook,” 3^{rd} Edition, Springer-Verlag, Berlin, (1997), p. 72, the entire teachings of which are incorporated herein by reference). About 1 equ. of NaH is add to a solution of 1R,2R-cyclohexanediol in dimethylformamide (DMF), followed by addition 4-(2-bromo-ethyl)-benzene to form a mixture of mono-ether, di-ether and unreacted starting material. This mixture is easily separated based on the differences in solubility and chromatographic properties of the constituents. The mono-ether product, 2R-(2-phenylethoxy)-cyclohexan-1R-ol, is dissolved in pyridine and an excess amount of mesyl chloride is added to the reaction mixture to form 1R-methanesulfonate-2R-(2-phenylethoxy)-cyclohexane. 1R-Methanesulfonate-2R-(2-phenylethoxy)-cyclohexane is dissolved in acetone and an excess amount of LiBr is added to achieve an SN² displacement of the methanesulfonate group to form 1S-bromo-2R-(2-phenylethoxy)-cyclohexane. 1S-Bromo-2R-(2-phenylethoxy)-cyclohexane and pyrrolidin-3R-ol are dissolved in acetonitrile in about equal molar amounts. The amino group of the pyrrolidin-3R-ol reacts in preference to the alcohol group to achieve an S_{N}2 displacement of the bromo group of 1S-bromo-2R-(2-phenylethoxy)-cyclohexane to form 1R-(3R-hydroxypyrrolidin-1-yl)-2R-(2-phenylethoxy)-cyclohexane.

Alternatively, 3R-benzyloxy-pyrrolidine can be used in the final reaction of Method I instead of pyrrolidin-3R-ol. 3R-Benzyloxy-pyrrolidine is prepared by treating pyrrolidin-3R-ol in a solution of THF with about 1 equ. of NaH. The reaction mixture is stirred for about 5 min. to about 30 min., then about 1 equ. of benzyl bromide is added to the reaction mixture. After stirring for about 2 hours, 3R-benzyloxy-pyrrolidine is formed.

When 3R-benzyloxy-pyrrolidine is used in the final reaction of Method I the benzyl protecting group is removed by dissolving the product in ethanol in the presence of a catalytic amount of palladium on carbon. The reaction is shaken under a hydrogen atmosphere to yield 1 R-(3R-hydroxypyrrolidin-1-yl)-2R-(2-phenylethoxy)-cyclohexane.

II. Method II for Preparing 1R-(3R-hydroxypyrrolidin-1-yl)-2R-(2-phenylethoxy)-cyclohexane.

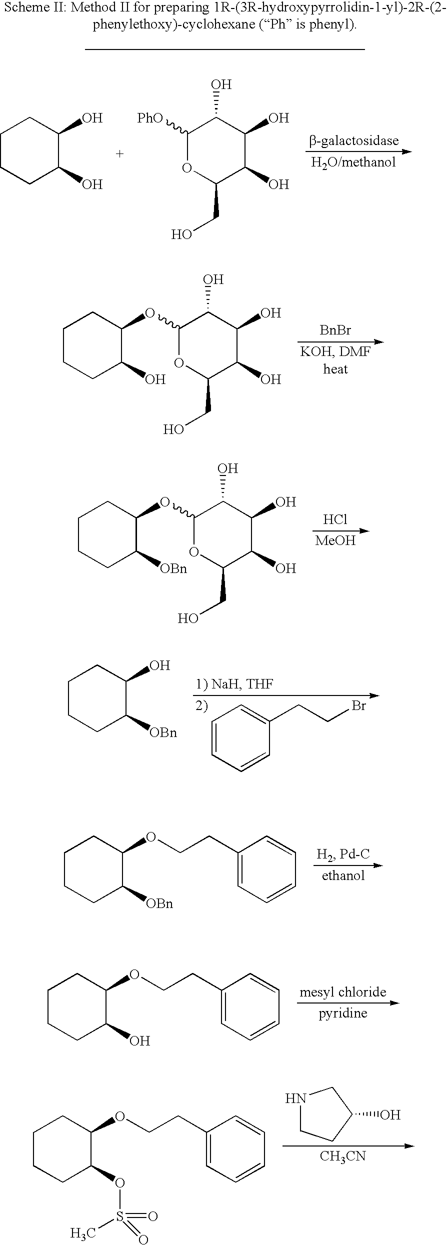

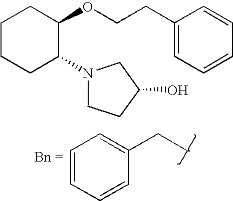

About equal molar amounts of meso-cis-1,2-cyclohexanediol and 1′-phenoxy-galactose are dissolved in a 1:1 mixture of water and methanol in the presence of a catalytic amount of β-galactosidase. β-galactosidase preferentially catalyzes reaction of the chiral carbon of meso-cis-1,2-cyclohexanediol having an R-configuration with 1′-O-phenyl-galactose. A benzyl protecting group is added to all free hydroxyl groups of the product by dissolving the product in dimethylformamide (DMF) in the presence of potassium hydroxide and an excess amount of benzyl bromide. The reaction mixture is typically heated to about 130° C. to about 140° C. The benzylated product is dissolved in a mixture of hydrochloric acid in methanol to cleave the glycosidic bond by acid hydrolysis, resulting in 2S-benzyloxy-cyclohexan-1R-ol. About 1 equ. of NaH is add to a solution of the 2S-benzyloxy-cyclohexan-1R-ol in THF. After the reaction mixture has stirred for about 5 min. to about 30 min., 4-(2-bromo-ethyl)-benzene is added to the reaction mixture to form 1S-benzyloxy-2R-(2-phenylethoxy)-cyclohexane. 1S-Benzyloxy-2R-(2-phenylethoxy)-cyclohexane is dissolved in ethanol in the presence of a catalytic amount of palladium on carbon. The reaction is shaken under a hydrogen atmosphere until the benzyl group is removed to form 2R-(2-phenylethoxy)-cyclohexan-1S-ol. 2R-(2-phenylethoxy)-cyclohexan-1S-ol is dissolved in pyridine, and an excess amount of mesyl chloride is added to the reaction mixture to form 1S-methanesulfonate-2R-(2-phenylethoxy)-cyclohexane. 1S-Methanesulfonate-2R-(2-phenylethoxy)-cyclohexane and pyrrolidin-3R-ol are dissolved in dichloromethane in about equal molar amounts. The amino group of the pyrrolidin-3R-ol reacts in preference to the alcohol group to achieve an S_{N}2 displacement of the methanesulfonate group to form 1R-(3R-hydroxypyrrolidin-1-yl)-2R-(2-phenylethoxy)-cyclohexane.

Alternatively, 3R-benzyloxy-pyrrolidine can be used in the final reaction of Method II instead of pyrrolidin-3R-ol. When 3R-benzyloxy-pyrrolidine is used in the final reaction of Method II, the benzyl protecting group is removed as described in Method I to yield 1R-(3R-hydroxypyrrolidin-1-yl)-2R-(2-phenylethoxy)-cyclohexane.

III. Method III for 1R-(3R-hydroxypyrrolidin-1-yl)-2R-(2-phenylethoxy)-cyclohexane.

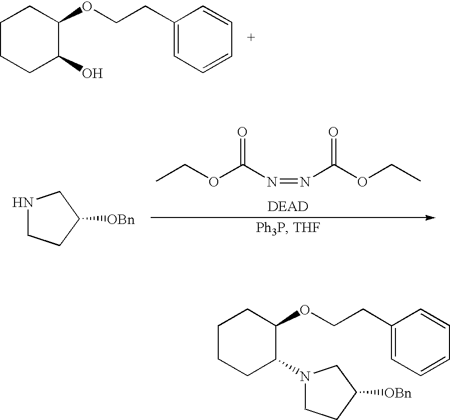

Scheme III: Method III for 1R-(3R-hydroxypyrrolidin-1-yl)-2R-(2-phenylethoxy)-cyclohexane.

2R-(2-phenylethoxy)-cyclohexan-1S-ol (prepared as in Method II), triphenyl phosphine (PPh₃), and diethylazodicarboxylate (DEAD) are dissolved in THF in about equal molar amounts with about 1.2 equ. to about 2 equ. of 3R-benzyloxy-pyrrolidine. After stirring at about −25° C. to about 25° C., 1R-(3R-benzyloxy-pyrrolidin-1-yl)-2R-(2-phenylethoxy)-cyclohexane is formed. The benzyl protecting group is removed to form 1R-(3R-hydroxypyrrolidin-1-yl)-2R-(2-phenylethoxy)-cyclohexane as described in Method I.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of stereoselectively preparing a 1,2-disubstituted cycloalkyl represented by the following structural formula: 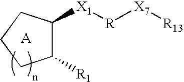
wherein:
ring A is substituted or unsubstituted;
n is 1, 2, or 3;
X₁ is —O—, —S—, or —NR₂—;
X₇ is a bond, —O—, —S—, or —CR₂₀═CR₂—;
R is an alkylene group;
R₁ is —OR₃, —SR₃ or —NR₄R₅;
R₁₃ is an aliphatic group, an aryl group or a heteroaryl group;
R₂ and R₃ are each, independently, —H, an aliphatic group, an aliphatic carbonyl, an aralkyl, an aryl, a heteroaryl, a heteroaralkyl, a heterocycloalkyl, or a group represented by the formula —Y—R₆, wherein Y is an alkylene group and R₆ is a heterocycloalkyl group;
R₄ and R₅ are each, independently, —H, an aliphatic group, an aliphatic carbonyl, an aralkyl, an aryl, a heteroaryl, a heteroaralkyl, a heterocycloalkyl, or a group represented by the formula —Y—R₆; or R₄ and R₅ together with the nitrogen to which they are attached is a heteroaryl or a heterocycloalkyl; and
R₂₀ and R₂₁ are each, independently, —H, an aliphatic group, an aryl group, or an aralkyl, comprising the following steps:
a) reacting a substituted cycloalkane represented by the following structural formula: 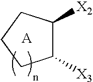
wherein:
X₂ is —OH, —SH, or —NHR₂; and
X₃ is —OH, a protected alcohol, or a halo,
with a compound having a leaving group represented by the following structural formula: 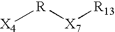
wherein X₄ is a leaving group, to form a compound represented by the following structural formula: 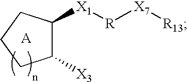
b) reacting the compound formed in step a) with:
i) a halogen source; or
ii) a carboxylic acid in the presence of triphenyl phosphine and dialkyl azodicarboxylate to form an ester, hydrolyzing the ester to form a hydroxy group and reacting the hydroxy group with a compound selected from the group consisting of X—SO₂-aryl, X—SO₂— (aliphatic group), and 2,2,2-trihaloacetonitrile,
to form a compound represented by the following structural formula: 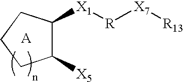
wherein X₅ is a halo, —OSO₂-aryl, —OSO₂— (aliphatic group), or 2,2,2-trihaloacetimidate; and
c) reacting the compound formed in step b) with a nucleophile selected from the group consisting of HR₁ or M⁺⁻R₁, wherein M⁺ is a metal cation, to form said 1,2-disubstituted cycloalkyl.

2. The method of claim 1, wherein X₂ and X₃ are —OH.

3. The method of claim 1, wherein X₄ is selected from the group consisting of a halo, —OSO₂-aryl, —OSO₂— (aliphatic group), and 2,2,2-trihaloacetimidate.

4. The method of claim 1, wherein X₃ is —OH and step b) comprises reacting the compound formed in step a) with a halogen source selected from the group consisting of SOCl₂ in pyridine and diarylchlorophosphite followed by treatment with HBr.

5. The method of claim 1, wherein X₃ is —OH and step b) comprises the steps of:
a) reacting the compound formed in step a) in the presence of an aprotic base with compound selected from the group consisting of X—SO₂-aryl, X—SO₂— (aliphatic group), and 2,2,2-trihaloacetonitrile, wherein X is a halo, to form an activated alcohol; and
b) reacting the activated alcohol with a halide salt.

6. The method of claim 5, wherein the halide salt is LiBr or LiCl.

7. The method of claim 1, wherein X₃ is a protected alcohol and step b) comprises the steps of:
a) removing the alcohol protecting group from the compound formed in step a) to form a deprotected alcohol; and
b) reacting the deprotected alcohol with a halogen source selected from the group consisting of SOCl₂ in pyridine and diarylchlorophosphite followed by treatment with HBr.

8. The method of claim 1, wherein X₃ is a protected alcohol and step b) comprises the steps of:
a) removing the alcohol protecting group from the compound formed in step a) to form a deprotected alcohol;
b) reacting the deprotected alcohol in the presence of a base with compound selected from the group consisting of X—SO₂-aryl, X—SO₂— (aliphatic group), and 2,2,2-trihaloacetonitrile, wherein X is a halo, to form an activated alcohol; and
c) reacting the activated alcohol with a halide salt.

9. The method of claim 8, wherein the halide salt is LiBr or LiCl.

10. The method of claim 2, wherein the nucleophile in step c) is HNR₄R₅.

11. The method of claim 10, wherein the nucleophile is a compound represented by the following structural formula: 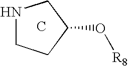
wherein:
R₈ is —H or an alcohol protecting group; and
ring C is substituted or unsubstituted.

12. The method of claim 11, wherein the compound having a leaving group in step
a) is a compound represented by the following structural formula: 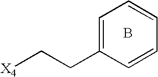
wherein ring B is substituted or unsubstituted.

13. The method of claim 12, wherein R₈ is —H.

14. The method of claim 13, wherein n is 2, X₁ is —O—, and the 1,2-disubstituted cycloalkyl formed is the compound represented by the following structural formula:

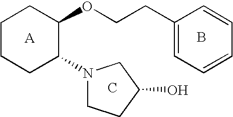

15. The method of claim 14, wherein the compound prepared is 1R-(3R-hydroxypyrrolidin-1-yl)-2R-(2-phenylethoxy)-cyclohexane.

16. A method of stereoselectively preparing a 1,2-disubstituted cycloalkyl represented by the following structural formula: 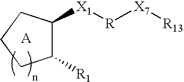
wherein:
ring A is substituted or unsubstituted;
n is 1, 2, or 3;
X₁ is —O—, —S—, or —NR₂—;
X₇ is a bond, —O—, —S—, or —CR₂₀═CR₂₁—;
R is an alkylene group;
R₁ is —OR₃, —SR₃ or —NR₄R₅;
R₁₃ is an aliphatic group, an aryl group or a heteroaryl group;
R₂ and R₃ are each, independently, —H, an aliphatic group, an aliphatic carbonyl, an aralkyl, an aryl, a heteroaryl, a heteroaralkyl, a heterocycloalkyl, or a group represented by the formula —Y—R₆, wherein Y is an alkylene group and R₆ is a heterocycloalkyl group;
R₄ and R₅ are each, independently, —H, an aliphatic group, an aliphatic carbonyl, an aralkyl, an aryl, a heteroaryl, a heteroaralkyl, a heterocycloalkyl, or a group represented by the formula —Y—R₆; or R₄ and R₅ together with the nitrogen to which they are attached is a heteroaryl or a heterocycloalkyl; and
R₂₀ and R₂, are each, independently, —H, an aliphatic group, an aryl group, or an aralkyl, comprising the following steps:
a) reacting in the presence of β-galactosidase a substituted cycloalkanol represented by the following structural formula: 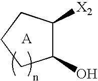
wherein X₂ is —OH, —SH, or —NHR₂,
with a galactose derivative represented by the following structural formula: 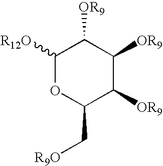
wherein:
R₉ for each occurrence is, independently, —H or an alcohol protecting group, or two adjacent —OR₉ groups together with the carbon atoms to which they are attached form a [1,3]dioxolane; and
R₁₂ is an aryl, a cycloalkyl, or a heterocycloalkyl,
to form a galactose substituted cycloalkanol represented by the following structural formula: 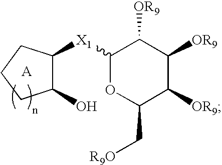
b) reacting the galactose substituted cycloalkanol with an alcohol protecting group to form a compound represented by the following structural formula: 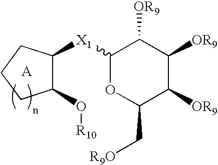
wherein R₁₀ is an alcohol protecting group;
c) treating the compound formed in step b) with an acid to form a protected cycloalkanol represented by the following structural formula: 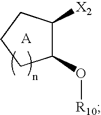
d) reacting the protected cycloalkanol with a compound having a leaving group represented by the following structural formula: 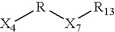
wherein X₄ is a leaving group, to form a compound represented by the following structural formula: 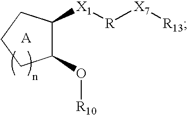
e) removing R₁₀ to form a compound represented by the following structural formula: 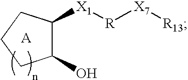
f) reacting the compound formed in step e) with an alcohol activating agent and a nucleophile selected from the group consisting of HR₁ or M⁺⁻R₁, wherein M⁺ is a metal cation, to form said 1,2-disubstituted cycloalkyl.

17. The method of claim 16, wherein X₂ is —OH.

18. The method of claim 17, wherein the compound having a leaving group in step d) is a compound represented by the following structural formula: 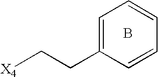
wherein ring B is substituted or unsubstituted.

19. The method of claim 18, wherein the alcohol activating agent in step f) includes a dialkyl azodicarboxylate and triphenyl phosphine, and the nucleophile is HNR₄R₅.

20. The method of claim 19, wherein the nucleophile is a compound represented by the following structural formula: 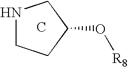
wherein:
R₈ is —H or an alcohol protecting group; and
ring C is substituted or unsubstituted.

21. The method of claim 18, wherein the alcohol activating agent in step f) is selected from the group consisting of X—SO₂— aryl, X—SO₂— (aliphatic group), and 2,2,2-trihaloacetonitrile, wherein X is a halo, and the nucleophile is HNR₄R₅.

22. The method of claim 21, wherein the nucleophile is a compound represented by the following structural formula: 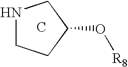
wherein:
R₈ is —H or an alcohol protecting group; and
ring C is substituted or unsubstituted.

23. The method of claim 22, wherein R₈ is —H.

24. The method of claim 23, wherein X, is —O—, n is 2, and the 1,2-disubstituted cycloalkyl formed is the compound represented by the following structural formula:

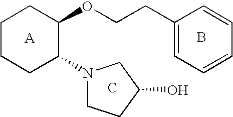

25. The method of claim 24, wherein the compound prepared is 1R-(3R-hydroxypyrrolidin-1-yl)-2R-(2-phenylethoxy)-cyclohexane.

26. A method of stereoselectively preparing a 1,2-disubstituted cycloalkyl represented by the following structural formula: 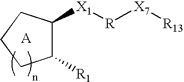
wherein:
ring A is substituted or unsubstituted;
n is 1, 2, or 3;
X₁ is —O—, —S—, or —NR₂—;
X₇ is a bond, —O—, —S—, or —CR₂₀═CR₂₁—;
R is an alkylene group;
R₁ is —OR₃, —SR₃ or —NR₄R₅;
R₁₃ is an aliphatic group, an aryl group or a heteroaryl group;
R₂ and R₃ are each, independently, —H, an aliphatic group, an aliphatic carbonyl, an aralkyl, an aryl, a heteroaryl, a heteroaralkyl, a heterocycloalkyl, or a group represented by the formula —Y—R₆, wherein Y is an alkylene group and R₆ is a heterocycloalkyl group;
R₄ and R₅ are each, independently, —H, an aliphatic group, an aliphatic carbonyl, an aralkyl, an aryl, a heteroaryl, a heteroaralkyl, a heterocycloalkyl, or a group represented by the formula —Y—R₆; or R₄ and R₅ together with the nitrogen to which they are attached is a heteroaryl or a heterocycloalkyl; and
R₂₀ and R₂₁ are each, independently, —H, an aliphatic group, an aryl group, or an aralkyl, comprising the following steps:
a) reacting in the presence of β-galactosidase a substituted cycloalkanol represented by the following structural formula: 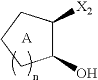
wherein X₂ is —OH, —SH, or —NHR₂,
with a galactose derivative represented by the following structural formula: 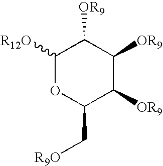
wherein:
R₉ for each occurrence is, independently, —H or an alcohol protecting group, or two adjacent —OR₉ groups together with the carbon atoms to which they are attached form a [1,3]dioxolane; and
R₁₂ is an aryl, a cycloalkyl, or a heterocycloalkyl, to form a galactose substituted cycloalkanol represented by the following structural formula: 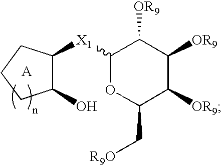
b) reacting the galactose substituted cycloalkanol with an alcohol activating agent and a nucleophile selected from the group consisting of HR₁ or M⁺⁻R₁, wherein M⁺ is a metal cation, to form a compound represented by the following structural formula: 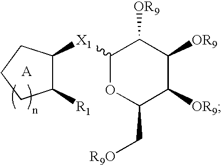
c) treating the compound formed in step b) with an acid to form a compound represented by the following structural formula: 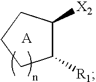
d) reacting the compound formed in step e) with a compound having a leaving group represented by the following structural formula: 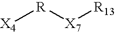
wherein X₄ is a leaving group, to form said 1,2-disubstituted cycloalkyl.

27. The method of claim 26, wherein X₂ is —OH.

28. The method of claim 27, wherein R₉ for each occurrence is —H.

29. The method of claim 28, further comprising the step of selectively protecting the hydroxy groups of the galactose substituent with a cyclic acetal or a cyclic ketal.

30. The method of claim 29, wherein the selectively protected hydroxy groups form an isopropylidene ketals.

31. The method of claim 30, wherein the compound having a leaving group in step d) is a compound represented by the following structural formula: 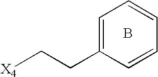
wherein ring B is substituted or unsubstituted.

32. The method of claim 31, wherein the alcohol activating agent in step b) includes a dialkyl azodicarboxylate and triphenyl phosphine, and the nucleophile is HNR₄R₅.

33. The method of claim 32, wherein the nucleophile is a compound represented by the following structural formula: 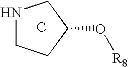
wherein:
R₈ is —H or an alcohol protecting group; and
ring C is substituted or unsubstituted.

34. The method of claim 31, wherein the alcohol activating agent in step b) is selected from the group consisting of X—SO₂— aryl, X—SO₂— (aliphatic group), and 2,2,2-trihaloacetonitrile, wherein X is a halo, and the nucleophile is HNR₄R₅.

35. The method of claim 34, wherein the nucleophile is a compound represented by the following structural formula: 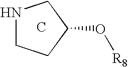
wherein:
R₈ is —H or an alcohol protecting group; and
ring C is substituted or unsubstituted.

36. The method of claim 35, wherein R₈ is —H.

37. The method of claim 36, wherein X₁ is —O—, n is 2, and the 1,2-disubstituted cycloalkyl formed is the compound represented by the following structural formula:

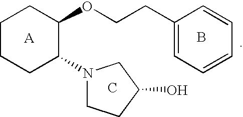

38. The method of claim 37, wherein the compound prepared is 1R-(3R-hydroxypyrrolidin-1-yl)-2R-(2-phenylethoxy)-cyclohexane.

39. A method of stereoselectively preparing a 1,2-disubstituted cycloalkyl represented by the following structural formula: 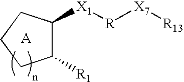
wherein:
ring A is substituted or unsubstituted;
n is 1, 2, or 3;
X, is —O—, —S—, or —NR₂—;
X₇ is a bond, —O—, —S—, or —CR₂₀=CR₂₁—;
R is an alkylene group;
R₁ is —OR₃, —SR₃ or —NR₄R₅;
R₁₃ is an aliphatic group, an aryl group or a heteroaryl group;
R₂ and R₃ are each, independently, —H, an aliphatic group, an aliphatic carbonyl, an aralkyl, an aryl, a heteroaryl, a heteroaralkyl, a heterocycloalkyl, or a group represented by the formula —Y—R₆, wherein Y is an alkylene group and
R₆ is a heterocycloalkyl group;
R₄ and R₅ are each, independently, —H, an aliphatic group, an aliphatic carbonyl, an aralkyl, an aryl, a heteroaryl, a heteroaralkyl, a heterocycloalkyl, or a group represented by the formula —Y—R₆; or R₄ and R₅ together with the nitrogen to which they are attached is a heteroaryl or a heterocycloalkyl; and
R₂₀ and R₂, are each, independently, —H, an aliphatic group, an aryl group, or an aralkyl, comprising the following steps:
a) reacting a substituted cycloalkane represented by the following structural formula: 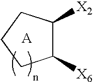
wherein:
X₂ is —OH, —SH, or —NHR₂; and
X₆ is a halo,
with a with a compound having a leaving group represented by the following structural formula: 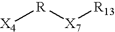
wherein X₄ is a leaving group, to form a compound represented by the following structural formula: 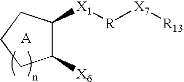
b) reacting the compound formed in step a) with HR₁ or M⁺⁻R₁, wherein M⁺ is a metal cation, to form said 1,2-disubstituted cycloalkyl.

40. The method of claim 39, wherein X₄ is selected from the group consisting of a halo, —OSO₂-aryl, —OSO₂— (aliphatic group), and 2,2,2-trihaloacetimidate.

41. The method of claim 39, wherein the nucleophile in step b) is HNR₄R₅.

42. The method of claim 41, wherein the nucleophile is a compound represented by the following structural formula: 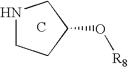
wherein:
R₈ is —H or an alcohol protecting group; and
ring C is substituted or unsubstituted.

43. The method of claim 42, wherein R₈ is —H.

44. The method of claim 43, wherein the compound having a leaving group in step a) is a compound represented by the following structural formula: 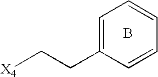
wherein ring B is substituted or unsubstituted.

45. The method of claim 44, wherein X, is —O—, X₂ is —OH and the 1,2-disubstituted cycloalkyl formed is the compound represented by the following structural formula:

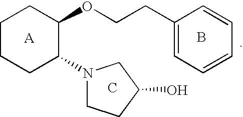

46. The method of claim 45, wherein the compound prepared is 1R-(3R-hydroxypyrrolidin-1-yl)-2R-(2-phenylethoxy)-cyclohexane.
